(19) United States
(12) Patent Application Publication
(10) Pub. No.: US 2024/0412810 A1
(43) Pub. Date: Dec. 12, 2024

(54) In Silico Generation of Binding Agents

(71) Applicant: Flagship Pioneering Innovations VI, LLC, Cambridge, MA (US)

(72) Inventor: John Ingraham, Cambridge, MA (US)

(21) Appl. No.: 18/694,894

(22) PCT Filed: Sep. 23, 2022
(86) PCT No.: PCT/US2022/076970

§ 371 Date: Mar. 22, 2024

Related U.S. Application Data
(60) Provisional application No. 63/261,646, filed on Sep. 24, 2021.

(51) Int. Cl. G16B 15/20; G16B 15/30; G16B 40/00

(52) U.S. Cl. CPC G16B 15/20; G16B 15/30; G16B 40/00

(57) ABSTRACT

In some embodiments, methods and corresponding systems are disclosed for providing associated biopolymer sequence(s) to conform to a reference structure. The reference structure includes a target complex and the one or more associated biopolymer sequences. The biopolymer sequences are obtainable by the method, including embedding a graph representation using a neural network. The graph representation is featurized from the reference structure and includes a topology of the biopolymer with monomers as nodes and interactions between monomers as edges. The methods, in certain embodiments, further include processing the graph representation with a graph neural network or equivariant neural network that iteratively updates node and edge embeddings with a learned parametric function. The methods may further include converting the embedded graph representation to an energy landscape using a decoder. The methods can further include obtaining one or more biopolymer sequences from the energy landscape.

### RELATED APPLICATION

This application claims the benefit of U.S. Provisional Application No. 63/261,646, filed on Sep. 24, 2021. The entire teachings of the above application are incorporated herein by reference.

### BACKGROUND

Biopolymers are fundamental building blocks of life and can serve both as targets for intervention and as effectors (such as therapeutics, e.g., antibodies, antibody-drug conjugates, fusion proteins, and aptamers). A common predicate for activity modulation is the ability of one or more biopolymers to form a complex through binding. Existing in silico modeling techniques typically are not geared to generating sequences of binders.

Accordingly, a need exists for systems and methods for in silico generation of binding agents (e.g., biopolymers).

### SUMMARY

Backbone structures of biopolymers (proteins, nucleic acids, carbohydrates, etc.) represent the physical shape of a biopolymer sequence (e.g., amino acid sequence, nucleotide sequence, sequence of carbohydrates). Biopolymer sequences can be represented as a sequence of monomers, and their backbone structures represent three-dimensional conformations of those sequences (e.g., when folded, when complexed with other biopolymers). Multiple backbone structures can interface with each other (e.g., antibodies and antigens). Existing methods for determining sequences based on backbone structures rely on physics-based models and search algorithms, which are typically cumbersome, slow, and inefficient.

In some embodiments, methods and corresponding systems are disclosed for providing associated biopolymer sequence(s) to conform to a reference structure. The reference structure includes a target complex. In embodiments, the reference structure can include one or more reference biopolymer sequences. The one or more associated biopolymer sequences are obtainable by the methods disclosed herein, including embedding a graph representation using a neural network. The graph representation is featurized from the reference structure and includes a topology of a biopolymer with monomers as nodes and interactions between monomers as edges. In embodiments, the graph representation can be featurized from the reference structure and includes a topology of a reference biopolymer, e.g., one or more reference biopolymer and/or one or more reference biopolymer sequences, with monomers as nodes and interactions between monomers as edges. The methods further include processing the graph representation with a graph neural network or equivariant neural network that iteratively updates node and edge embeddings with a learned parametric function. The methods further include converting the embedded graph representation to a conditional generative model using a decoder. The methods further include obtaining one or more associated biopolymer sequences from the conditional generative model.

In some embodiments, the target complex of the reference structure is a backbone structure copied from an experimentally determined structure (e.g., a crystal structure, such as an X-ray crystal structure or a NMR structure or a cryo-EM structure) as a template. In some embodiments, the target complex of the reference structure uses structure modeling to create a new backbone structure in silico. In some embodiments, a hybrid approach of using known/experimentally determined backbone structures and modeled backbone structures (e.g., in silico generated backbone structures), such as designing part of a backbone structure of a biopolymer sequence, but leaving a portion of the experimentally derived portion intact.

The biopolymers can include proteins, non-protein biopolymers (e.g., nucleic acids (aptamers)), and carbohydrate polymers, as well as combinations of the forgoing, as well as non-naturally occurring biopolymers—e.g., d-proteins, locked nucleic acids, peptide nucleic acids, etc. In addition, the biopolymers can be branched biopolymers or linear biopolymers. The biopolymers can comprise canonical monomers, non-canonical monomers, and combinations of both canonical and non-canonical monomers.

In some embodiments, the conditional generative model is an energy landscape or energy based model. Conditional generative models are trained to generate samples similar to a data distribution, e.g., by modeling the joint or conditional distributions of data. Parametric models are trained to generate samples similar to a data distribution, usually by modeling the joint or conditional distributions of data. Therefore, conditional generative models are generative models that are trained to estimate how to conditionally generate samples from input data. In this case, the input data is backbone structures of a protein complex, for example, backbone structures in which some or all of the R-groups of the amino acids in the proteins are omitted. Examples of generative models that can be trained in this conditional manner include Site-independent models, Potts models, Variational Autoencoders (VAEs), Generative Adversarial Networks (GANs), Autoregressive likelihood models.

In some embodiments, the energy landscape is a conditional random field representing the target complex and the one or more associated biopolymer sequences.

In some embodiments, obtaining the one or more associated biopolymer sequences from the energy landscape employs a maximum likelihood method.

In some embodiments, obtaining the one or more associated biopolymer sequences from the energy landscape employs an energy minimization process. In some embodiments, the energy minimization process employs a Monte Carlo simulation, annealing, integer-linear programming, or continuous relaxation-based optimization.

In some embodiments, the decoder is a generative model or a conditional generative model selected from one of the following:
a) a site-independent model predicting the marginal probability of each possible monomer at each position,
b) a conditional random field layer, or Potts model, with pairwise couplings between monomers,
c) an energy-based model with higher order interactions and/or a neural network parameterization
d) an autoregressively factorized language model,
e) a continuous latent variable model modeling, potentially structured as a variational autoencoder,
f) a discrete latent variable model, or
g) an implicit generative model.

The above listing are examples of generative models for generating sequences (e.g. sequences of words or biological sequences in our case) as a sequence of decisions, where each decision is modeled as dependent on the prior decisions. In the case of natural language, these are models that predict each word in a document given all of the preceding words (e.g., Generative Pre-trained Transformer 3 (GPT3) is one example used for natural language generation). In the present disclosure, the above models predict each monomer type at each position in the structure as a sequence of decisions conditioned on previous or preceding decisions. This notion of “preceding” can be generalized, such that the preceding or previous entry is not literally in left-to-right western reading order, as in the natural language processing case. Rather, autoregressive models simply predict the items in an object as a sequence of decisions in some predetermined order.

In some embodiments, the decoder is structured as a conditional random field. In some embodiments, the conditional random field is parameterized by a first term and a second term, the first term representing a monomer bias at each position in the reference structure and the second term representing interdependencies between monomers in the structure. In some illustrative embodiments, the one or more associated biopolymer sequence is a protein and the conditional random field is characterized by

$$P ( {s}_{1} , … , {s}_{N} \vert X ) = \frac{1}{Z} exp \{ - {\sum }_{i} {h}_{i} [ {s}_{i} ; X ] - {\sum }_{i < j} {J}_{ij} [ {s}_{i} , {s}_{j} ; X ] \} ,$$

wherein sᵢ refers to the monomer identity at position i, X refers to the entire backbone structure of the reference structure, hᵢ[sᵢ; X] refers to the bias term for monomer type sᵢ at position i that is output by the network given X, and Jᵢⱼ[sᵢ, sⱼ; X] refers to the coupling term between monomer type sᵢ at position i and monomer type sⱼ at position j. This can be applied analogously to non-protein biopolymers.

In some embodiments, the target complex comprises one or more reference biopolymer sequences. In some embodiments, the target complex comprises the biopolymer, i.e., the biopolymer for which the topology is included in the graph representation.

In some embodiments, the target complex comprises at least one molecule that is not a biopolymer.

In some embodiments, the reference structure is a complex of two or more reference biopolymers. In some embodiments, obtaining the one or more biopolymer sequence from the energy landscape further includes obtaining one or more biopolymer sequences relating to binding the target complex comprising two or more biopolymer sequences.

In some embodiments, the topology of monomers comprises a representation of one or more (e.g., 1, 2, 3, 4, 5, 6, or all 7) of bond lengths, bond angles, dihedral angles, scalar lengths and angles as vectorial values through radial basis functions, angular embeddings, and at least one categorical discretization.

In some embodiments, the topology is based on k-nearest neighbors, wherein k is about: 10, 15, 20, 25, 30, 35, 40, 45, 50, or more.

In some embodiments, the topology is based on monomer centroid distance of about: 5, 6, 7, 8, 9, 10, 11, 12, 13, 14, 15, 16, 17, 18, 19, or 20 angstroms, or more. In some embodiments, the biopolymer, i.e., reference biopolymer, is a protein and the monomer centroid is the alpha-carbon of amino acids in the protein.

In some embodiments, the edges comprise one or more of (e.g., 1, 2, 3, or all 4):
a) primary sequence distance between monomers,
b) whether the pairs of monomers are in the same or different polymers in the reference structure, interatomic distances between monomers,
c) relative orientations of atoms at the first monomer i and atoms at the second monomer j, for example the relative location of atoms at the second monomer j when canonicalized in a reference frame based on first monomer i, and
d) raw Cartesian displacements between atoms at the first monomer i and the second monomer j.

In some embodiments, the methods are for providing a full chain design for the one or more associated biopolymer sequences to conform to the reference structure, the reference structure including at least one of a structure formed by naturally occurring sequences, structures formed by an in silico generated sequence, and structures generated in silico unassociated with a sequence.

In some embodiments, the methods are for providing a design of interfacial monomers of the one or more associated biopolymer sequences to conform to the reference structure.

In some embodiments, the methods are for providing a design of surface monomers of the one or more associated biopolymer sequences to conform to the reference structure.

In some embodiments, the methods are for providing the one or more associated biopolymer sequences to conform to the reference structure using a limited set of monomers.

In some embodiments, the reference structure comprises a backbone of the one or more reference biopolymer sequences. In some embodiments, the backbone omits some or all of the side chains of the one or more reference biopolymer sequences. In some embodiments, the reference structure comprises a backbone of the biopolymer, i.e., the biopolymer for which the topology is included in the graph representation.

In some embodiments, the methods further include concurrently or sequentially altering the one or more associated biopolymer sequences to modulate one or more biophysical properties or pharmacodynamic properties of the associated biopolymer sequences, the one or more biophysical properties or pharmacodynamic properties selected from: isoelectric point, weight, hydrophobicity, melting temperature, stability, Kₒₙ, K_{off}, or K_{d}, half-life, enzymatic function, aggregation, and functional activity.

In some embodiments, the one or more associated biopolymer sequences is a polypeptide. In some embodiments, the polypeptide comprises one or more non-canonical amino acids. In some embodiments, the polypeptide comprises one or more D-amino acids. In some embodiments, the polypeptide is an antibody or antigen-binding fragment thereof and the reference structure is an antibody-antigen complex. In some embodiments, the polypeptide is a ligand or receptor, and the reference structure is a ligand-receptor complex. In some embodiments, the polypeptide is an enzyme or substrate, and the reference structure is an enzyme-substrate complex. Antigens, ligands, and substrates can include both naturally occurring antigens, ligands, and substrates, as well as artificially designed antigens, ligands, and substrates, e.g., ones engineered to modulate activity, such as agonists, antagonists; either of which may be partial or complete and which may or may not induce biased signaling modulation.

In some embodiments, the methods can provide one or more n-mer biopolymer sequences in under about: 120, 60, 30, 10, 9, 8, 7, 6, 5, 4, or 3 seconds, wherein n is greater than about: 100, 200, 300, 400, or 500. In one illustrative exemplification, when used to redesign arbitrary subsystems (e.g., the interface, any chain, or all chains at the same time) of the antibody: lysozyme complex “1FDL”, which contains 561 crystallized residues, the methods (and/or associated systems) can do this in about 2.8 seconds using 100 Monte Carlo sweeps on a 2.6 GHz 6-Core Intel Core i7 made in 2019.

In some embodiments, the one or more associated biopolymer sequences is a protein and wherein the model was trained: using an ensemble of about: 1000, 2000, 3000, 5000, 10000, 50000, 100000, 500000, 1000000, or more, protein structures, e.g., some (e.g., 5, 10, 20, 30, 40, 50, 60, 70, 80, 90, 95%) or substantially all of Protein Data Bank (PDB).

In some embodiments, the methods are configured to provide training on the target complex, wherein the target complex involves multiple chains. In this embodiment, the methods use data with multiple chains at training time and, optionally, includes the features that distinguish the multiple chains from different polymers.

In some embodiments, the one or more associated biopolymer sequences are proteins and the energy landscape is a conditional random field such as a Potts model.

In some embodiments, edges are initialized using edge features based on the geometric and structural relationships between the biopolymer, i.e., the biopolymer for which the topology is included in the graph representation.

In some embodiments, methods and corresponding systems are disclosed for providing one or more associated biopolymer sequences to conform to a reference structure. The reference structure includes a target complex. In embodiments, the reference structure includes a target complex and one or more reference biopolymer sequences. The one or more associated biopolymer sequences are obtainable by the methods including obtaining a first biopolymer sequence from an energy landscape, where the energy landscape is generated based on a graph representation embedded using a neural network. The graph representation is featurized from the reference structure and comprising a topology of biopolymer sequences as nodes and interactions between monomers as edges. The methods further include generating one or more additional biopolymer sequences using the energy sequence, free of using the graph representation.

In some embodiments, the methods include synthesizing the one or more additional biopolymer sequences. Embodiments can include synthesizing the one or more associated biopolymer sequences.

In some embodiments, the methods include contacting the one or more additional biopolymer sequences with an analyte, e.g., a biological fluid or test sample.

In some embodiments, the methods include producing one or more additional biopolymer sequences obtainable by any one of the foregoing methods, systems, etc., optionally wherein the one or more biopolymer sequences may be conjugated to an additional moiety. In some embodiments, the biopolymer sequence is an antibody.

In some embodiments, the methods include administering to a subject in need a particular biopolymer sequence (or a conjugate comprising the same), the particular biopolymer sequence producible by any one of the foregoing claims.

In another aspect, a non-transient, computer-readable medium comprising instructions to be performed by a microprocessor, suitable for performing any one of the foregoing methods is provided.

In some embodiments, the systems comprise the non-transient, computer-readable medium disclosed above, and a processor.

In some embodiments, an associated biopolymer sequence is a unique sequence, ensemble of sequences, or distribution of sequences probabilities (e.g., at a given position in a chain).

In some embodiments, a polypeptide is produced (or is producible) by the above methods. The polypeptide can be an antibody.

Embodiments can start with a target structure, and using the methods and systems described herein, produce sequences that are predicted to fold to this target structure. In embodiments, the target structure that is the input can be the structure of a native protein, in which case there is an associated native (reference) sequence. In embodiments, the target structure can be totally made-up, e.g., a hypothetical structure one would like to achieve. In the example of a made-up structure, there is not an associated reference sequence. In embodiments, a reference sequence can be associated with the target structured to perform constrained optimization by varying only a part of the reference versus generating an entirely new sequence from scratch.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 is a flow diagram illustrating an example embodiment of the present disclosure.

FIG. 2A is a diagram illustrating three components of the architecture of an example embodiment.

FIG. 2B is a diagram illustrating an example embodiment of node embeddings.

FIG. 2C is a diagram illustrating an example embodiment of edge embeddings.

FIGS. 3A-B are graphs illustrating the performance of the present method.

FIG. 4 illustrates a computer network or similar digital processing environment in which embodiments of the present invention may be implemented.

FIG. 5 is a diagram of an example internal structure of a computer (e.g., client processor/device 50 or server computers 60) in the computer system of FIG. 4.

### DETAILED DESCRIPTION

A description of example embodiments follows.

In some embodiments, herein is disclosed systems and corresponding methods for generating novel functional sequences of a biopolymer (e.g., protein), such as a biopolymer in a complex (e.g., biopolymers that are physically associated, at least in part, through non-covalent interactions, such as quaternary complexes, antibody-antigen, receptor-ligand, enzyme-substrate, etc.) of two or more biopolymers, given the three-dimensional structure of its backbone (e.g., the three-dimensional structure of the biopolymer when present in a complex of two or more biopolymers) is provided. The system and corresponding method, in some embodiments, comprises (i) a machine learning model that is trained end-to-end to predict a distribution over possible sequences given a protein structure and (ii) a design method that generates new sequences optimizing probability of the sequences matching the backbone structure of the complex, optionally subject to constraints on some monomers (e.g., residues). This method can be used for generating new interfaces between biopolymers within complexes, resurfaced biopolymers, fully redesigned sequences of experimentally measured structures, or fully de novo biopolymers based on computationally generated backbones because any arbitrary subset of monomers can be designed. These advantageous methods are based, at least in part, by introducing a model design that admits fast and constrained optimization (e.g., a conditional Potts model) and new flexible representations (e.g., of architecture and features) of full complete biopolymers, including biopolymers in complexes.

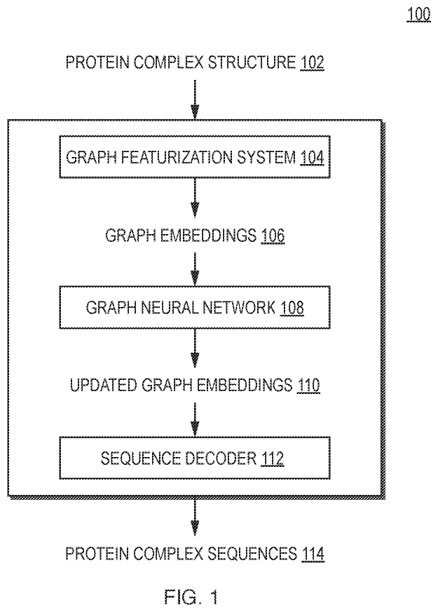

FIG. 1 is a flow diagram illustrating an example embodiment of the present disclosure. In the below example, the biopolymer being used as input is a protein. A protein complex structure 102 is input to the system and is processed by a graph featurization system 104. The graph featurization system generates graph embeddings 106, which characterize the backbone of the protein complex structure with nodes representing placement of molecules and edges representing connections between the molecules. A graph neural network 108 updates the embeddings and generates updated graph embeddings 110 as an energy field. A sequence decoder 112 then generates the biopolymers and outputs protein complex sequences 114.

In some embodiments, the disclosed system and methods train a neural network to directly generate biopolymer sequences given the 3D structure of the backbones in a biopolymer complex. This is framed as a conditional generative modeling problem, where the conditional distribution P(sequence|structure) is parameterized with a deep neural network and this model is trained end-to-end on biopolymer structure data to maximize likelihood.

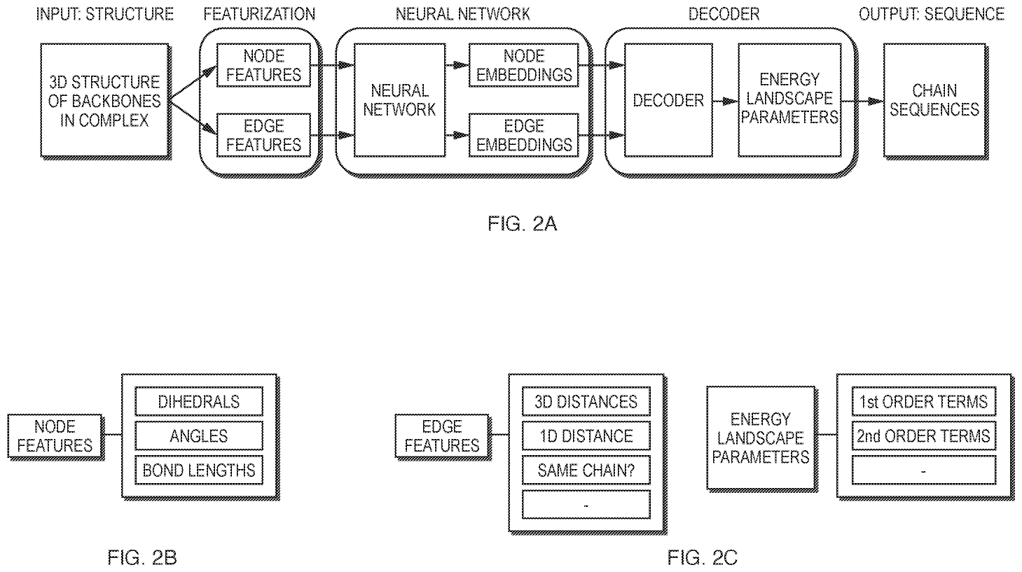

FIG. 2A is a diagram illustrating three components of the architecture. First, the backbone biopolymer structure is represented in terms of monomer-wise (e.g., node) features and/or monomer pair-wise (e.g., edge) features capturing aspects of the local and pairwise geometries of the backbone (e.g., illustrated further in FIG. 2B). The model is trained to predict P(sequence|structure) directly from structural coordinates through a series of differentiable modules. Second, a deep neural network processes these node and edge features into embeddings that can capture a combination of the local and broader geometric context for each monomer (e.g., node embeddings) and/or monomer pair (e.g., edge embeddings). Input features are comprised of a variety of geometric representations of biopolymer structure. Third, a decoder module converts these node and edge embeddings into the parameters of an energy landscape, which in turn defines P(sequence|structure). Energy landscape parameters consist of site and pairwise constraints on the sequence. The energy landscape, in some embodiments, is a conditional generative model for sequences

The graph featurization of FIG. 2A is described in further detail. The first step of the system is to process an input structure into a graph representation. This representation includes (1) a graph topology in which nodes in the graph correspond to monomers in the biopolymer complex and edges represent relationships between monomers, and (2) graph embeddings, which are vector encodings of information at each node (node embedding) and edge (edge embedding). This graph representation is further processed by the graph neural network and is initialized using geometric and other relational features that are computed from the input structure. Novel features of processing the graph representation include (a) new feature representations capturing more detailed atomistic geometry information and (b) features to seamlessly allow training on protein complexes involving multiple states.

The graph topology may, in some embodiments, be built as the k-Nearest Neighbors graph based on the backbone atoms in the protein complex, for example, the 30-nearest neighbors as measured by C-alpha backbone atom distance. The topology may alternatively be defined by a cutoff distance, such as including edges for all pairs of atoms whose C-alpha distances are less than 10 angstroms.

FIG. 2B illustrates an example embodiment of node embeddings. Node embeddings may be initialized from node features based on the geometry of the protein backbones. For example, the bond lengths, bond angles, and dihedral angles of the backbone may be represented as vectors and added to the initial node embeddings. In embodiments, not all biopolymers have dihedral angles, such as polypeptides, carbohydrates. In some embodiments, scalar lengths and angles as vectorial values through radial basis functions, angular embeddings, and at least one categorical discretization may be represented as vectors and added to the initial node embeddings as well. The angular features may be represented as points on the unit circle before embedding into the dimension of the node embeddings.

FIG. 2C illustrates an example embodiment of edge embeddings. Edge embeddings may be initialized from edge features based on the geometric and structural relationships between amino acids. These features can be based on:
a) the encoding of primary sequence distance between monomers,
b) the encoding of whether the pair of monomers are in the same or different chains,
c) the interatomic distances between monomers [e.g. 8×8 matrix of distances containing four backbone atoms at residues i and j],
d) the relative orientations of atoms at i and atoms at j, for example the relative location of atoms at monomer j when canonicalized in the frame of the monomer at I,
e) raw Cartesian displacements between atoms at i and j, to be used with for example equivariant Graph neural network layers.

The graph neural network may process the node and edge features, where the node embeddings and edge embeddings are both updated in a message-passing process. The updated node and edge embeddings may serve as input to a conditional random field decoder in the sequence generation layer.

A sequence decoder may be a generative model (e.g., a generative neural network or GNN) for generating sequences given the node and edge embeddings in the model, including
a) A site-independent model predicting the marginal probability of each possible monomer at each position,
b) A conditional random field layer (e.g., Potts model) with pairwise couplings between monomers,
c) An autoregressive decoding language model (Ingraham et al 2019),
d) A variational autoencoder for the conditional joint configuration of all monomers in the biopolymer.

In some embodiments, the sequence decoder can employ a conditional random field. An element of the present disclosure is that the decoder module is structured as a conditional random field, which can also be referred to as a conditional Potts model or conditional energy function. This conditional output distribution is parameterized by first and second terms that capture the sequence biases at each position in the structure as well as the interdependencies between positions. In embodiments, the conditional output distribution can be extended to higher-order terms. The conditional distribution can then be represented by the following relationship,

$$P ( {s}_{1} , … , {s}_{N} \vert X ) = \frac{1}{Z} exp \{ - {\sum }_{i} {h}_{i} [ {s}_{i} ; X ] - {\sum }_{i < j} {J}_{ij} [ {s}_{i} , {s}_{j} ; X ] \} , \qquad ( 1 )$$

where sᵢ refers to the monomer or rotamer identity at position i, X refers to the entire backbone structure of the input complex, hᵢ[sᵢ; X] refers to the bias term for letter or rotamer sᵢ at position i that is output by the network given X, and Jᵢⱼ[sᵢ, sⱼ; X] refers to the coupling term between letter or rotamer sᵢ at position i and letter sⱼ at position j.

The model may be trained on a collection of structures of diverse biopolymer complexes, for example, for proteins, from the Protein Data Bank. The protein complex dataset may be further processed to reduce redundant representations of certain sequence clusters, as well as to overrepresent protein complexes of interest such as protein therapeutic: target co-crystal structures. During training, data augmentation may be used, for example by adding noise to the input structures or replacing sequences with homologous sequences from genetic databases.

In some embodiments, the methods can be optimized with a conditional random field. After running the network once on a biopolymer to compute the parameters of the conditional random field, the intermediate computation of the graph network may be discarded and the energy landscape can be used to generate the sequence. Generating sequences with high probability P(s₁, . . . , s_{N}|X) reduces to minimizing the energy Σᵢhᵢ[sᵢ; X]+Σ_{i<j}Jᵢⱼ[sᵢ, sⱼ; X], which can straightforwardly be accomplished with methods known to a person having ordinary skill in the art, such as Monte Carlo simulated annealing or integer-linear programing.

In some embodiments, a partial design of subsequences can be accomplished using a conditional random field. Conditioning distributions of the form above (Equation 1) to account for specific residue constraints is simple; it suffices to simply restrict the domain of the sampling or optimization algorithm to account for the constraint. Thus, the allowed residues at each position can be set arbitrarily to either account for a known sequence or a required sub-set of allowed amino acids.

In some embodiments, the model of the present disclosure can be applied to design any or all of the sequence in a biopolymer complex given a model of the backbone 3D structure. Some relevant problems that fit this specification include
a) Full chain design-designing a complete biopolymer sequence given the structure,
b) Interface design-design the interfacial monomers given the biopolymer complex backbone,
c) Surface redesign-design the surface monomers of a biopolymer given the entire structure,
d) Restricted alphabet design-Redesign a sequence while restricting the alphabet to a subset of monomers given the structure,
e) Full de novo design-generate sequences from backbone structures that were generated by another computational method.

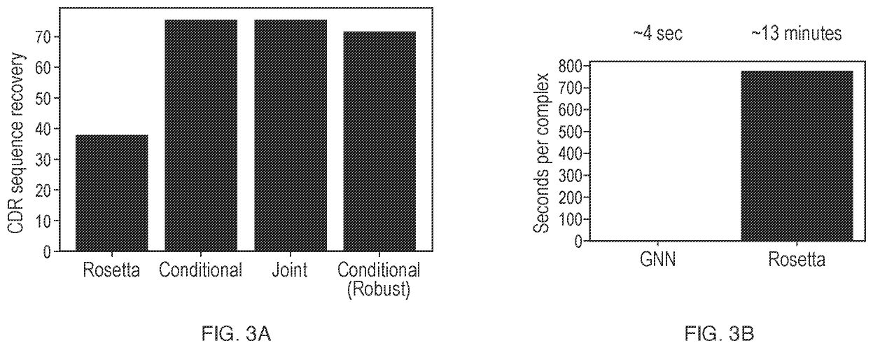

FIGS. 3A-B are graphs illustrating the performance of the present method. In a first graph of FIG. 3A, Applicant's methods (conditional, joint, conditional (robust)) are shown to recover more CDR sequences than the Rosetta method. In a second graph of FIG. 3B, Applicant's GNN methods are shown to generate a sequence in 4 seconds, while the Rosetta method takes around 13 minutes to generate a sequence. Therefore, a clear performance gain is shown by Applicant's disclosure.

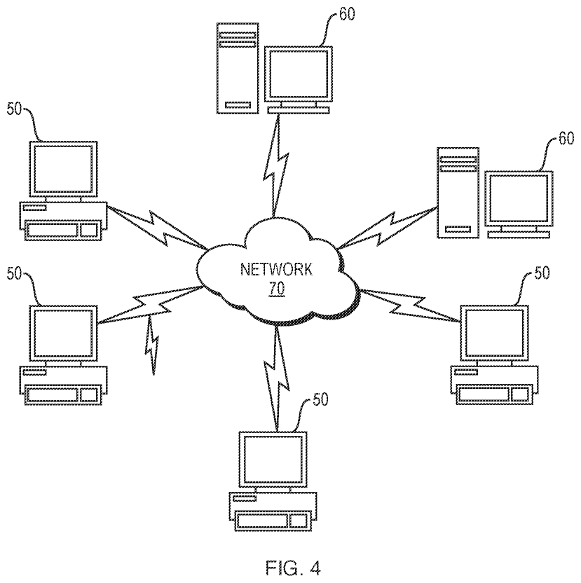

FIG. 4 illustrates a computer network or similar digital processing environment in which embodiments of the present invention may be implemented.

Client computer(s)/devices 50 and server computer(s) 60 provide processing, storage, and input/output devices executing application programs and the like. The client computer(s)/devices 50 can also be linked through communications network 70 to other computing devices, including other client devices/processes 50 and server computer(s) 60. The communications network 70 can be part of a remote access network, a global network (e.g., the Internet), a worldwide collection of computers, local area or wide area networks, and gateways that currently use respective protocols (TCP/IP, Bluetooth®, etc.) to communicate with one another. Other electronic device/computer network architectures are suitable.

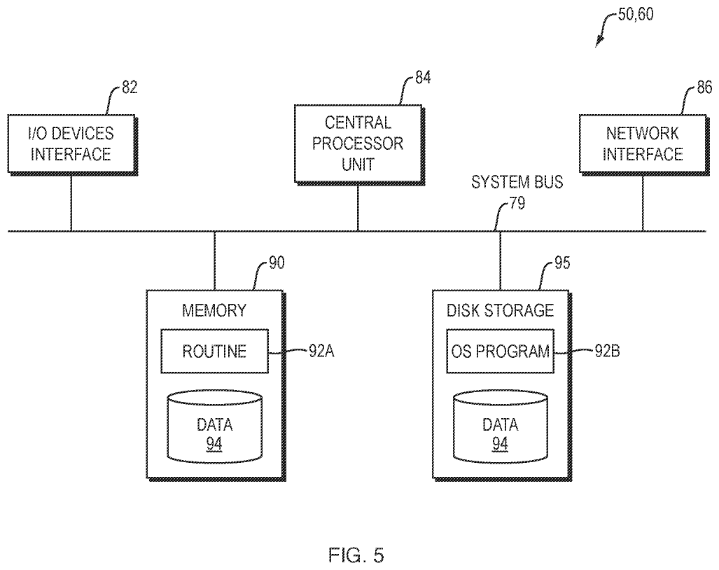

FIG. 5 is a diagram of an example internal structure of a computer (e.g., client processor/device 50 or server computers 60) in the computer system of FIG. 4. Each computer 50, 60 contains a system bus 79, where a bus is a set of hardware lines used for data transfer among the components of a computer or processing system. The system bus 79 is essentially a shared conduit that connects different elements of a computer system (e.g., processor, disk storage, memory, input/output ports, network ports, etc.) that enables the transfer of information between the elements. Attached to the system bus 79 is an I/O device interface 82 for connecting various input and output devices (e.g., keyboard, mouse, displays, printers, speakers, etc.) to the computer 50, 60. A network interface 86 allows the computer to connect to various other devices attached to a network (e.g., network 70 of FIG. 5). Memory 90 provides volatile storage for computer software instructions 92 and data 94 used to implement one or more embodiment of the present invention (e.g., machine learning modules, neural networks, GNNs, Conditional Generative Networks, and other networks disclosed above). Disk storage 95 provides non-volatile storage for computer software instructions 92 and data 94 used to implement an embodiment of the present invention. A central processor unit 84 is also attached to the system bus 79 and provides for the execution of computer instructions.

In one embodiment, the processor routines 92 and data 94 are a computer program product (generally referenced 92), including a non-transitory computer-readable medium (e.g., a removable storage medium such as one or more DVD-ROM's, CD-ROM's, diskettes, tapes, etc.) that provides at least a portion of the software instructions for the invention system. The computer program product 92 can be installed by any suitable software installation procedure, as is well known in the art. In another embodiment, at least a portion of the software instructions may also be downloaded over a cable communication and/or wireless connection. In other embodiments, the invention programs are a computer program propagated signal product embodied on a propagated signal on a propagation medium (e.g., a radio wave, an infrared wave, a laser wave, a sound wave, or an electrical wave propagated over a global network such as the Internet, or other network(s)). Such carrier medium or signals may be employed to provide at least a portion of the software instructions for the present invention routines/program 92.

While example embodiments have been particularly shown and described, it will be understood by those skilled in the art that various changes in form and details may be made therein without departing from the scope of the embodiments encompassed by the appended claims.

1. A method comprising providing one or more associated biopolymer sequences to conform to a reference structure, the reference structure comprising a target complex, the associated biopolymer sequences obtainable by a method comprising:
embedding a graph representation using a neural network, the graph representation featurized from the reference structure and comprising a topology of a biopolymer with monomers as nodes and interactions between monomers as edges;
processing the graph representation with a graph neural network or equivariant neural network that iteratively updates node and edge embeddings with a learned parametric function;
converting the embedded graph representation to an energy landscape using a decoder; and
obtaining one or more associated biopolymer sequences from the energy landscape.

2. The method of claim 1, wherein the energy landscape is a conditional generative model for sequences.

3. The method of claim 1, wherein the energy landscape is a conditional random field representing the target complex and the one or more associated biopolymer sequences.

4. The method of claim 1, wherein obtaining the one or more associated biopolymer sequences from the energy landscape employs a maximum likelihood method.

5. The method of claim 1, wherein obtaining the one or more associated biopolymer sequences from the energy landscape employs an energy minimization process.

6. The method of claim 5, wherein the energy minimization process employs a Monte Carlo simulation, simulated annealing, integer-linear programming, genetic process, variational inference, or continuous relaxation based optimization.

7. The method of claim 1, wherein the decoder is a generative model or a conditional generative model selected from at least one of the following:
a site-independent model predicting marginal probability of each possible monomer at each position,
a conditional random field layer, or Potts model, with pairwise couplings between monomers,
an energy-based model with at least one of higher order interactions and a neural network parameterization,
an autoregressively factorized language model,
a continuous latent variable model,
a discrete latent variable model, and
an implicit generative model.

8. The method of claim 1, wherein the decoder is structured as a conditional random field.

9. The method of claim 8, wherein the conditional random field is parameterized by a first term and a second term, the first term representing a monomer bias at each position in the reference structure and the second term representing interdependencies between monomers in the reference structure.

10. The method of claim 9, wherein the one or more associated biopolymer sequence is a protein and the conditional random field is characterized by
$$P ( {s}_{1} , … , {s}_{N} \vert X ) = \frac{1}{Z} exp \{ - {\sum }_{i} {h}_{i} [ {s}_{i} ; X ] - {\sum }_{i < j} {J}_{ij} [ {s}_{i} , {s}_{j} ; X ] \}$$
wherein s refers to monomer identity at position i, X refers to an entire backbone structure of the reference structure, hᵢ[sᵢ; X] refers to a bias term for monomer type sᵢ at position i that is output by the network given X, and Jᵢⱼ[sᵢ,sⱼ; X] refers to a coupling term between monomer type sᵢ at position i and monomer type sⱼ at position j.

11. The method of claim 1, wherein the target complex comprises the biopolymer.

12. The method of claim 1, wherein the target complex comprises a molecule that is not a biopolymer.

13. The method of claim 1, wherein the target complex is a complex comprising two or more reference biopolymer sequences.

14. The method of claim 13, wherein obtaining the one or more associated biopolymer sequences from the energy landscape further includes obtaining a given associated biopolymer sequences relating to binding the target complex comprising the two or more reference biopolymer sequences.

15. The method of claim 1, wherein the topology comprises a representation of one or more of bond lengths, bond angles, dihedral angles, scalar lengths and angles as vectorial values through radial basis functions, angular embeddings, and at least one categorical discretization.

16. The method of claim 1, wherein the topology is based on k-nearest neighbors, wherein k is about: 10, 15, 20, 25, 30, 35, 40, 45, or 50.

17. The method of claim 1, wherein the topology is based on monomer centroid distance of about: 5, 6, 7, 8, 9, 10, 11, 12, 13, 14, 15, 16, 17, 18, 19, or 20 angstroms.

18. The method of claim 17, wherein the biopolymer is a protein and the monomer centroid is an alpha-carbon of amino acids in the protein.

19. The method of claim 1, wherein the edges comprise one or more of: primary sequence distance between monomers, wherein pairs of monomers are in a same or different polymers in the reference structure, interatomic distances between monomers, relative orientations of atoms at a first monomer i and atoms at a second monomer j, and raw Cartesian displacements between atoms at the first monomer i and the second monomer j.

20. The method of claim 1, further comprising: providing a full chain design for the one or more associated biopolymer sequences to conform to the reference structure, the reference structure including at least one of a structure formed by naturally occurring sequences, structures formed by an in silico generated sequence, and structures generated in silico unassociated with a sequence.

21. The method of claim 1, further comprising: providing a design of interfacial monomers of the one or more associated biopolymer sequences to conform to the reference structure.

22. The method of claim 1, further comprising: providing a design of surface monomers of the one or more associated biopolymer sequences to conform to the reference structure.

23. The method of claim 1, further comprising: providing the one or more associated biopolymer sequences to conform to the reference structure using a limited set of monomers.

24. The method of claim 1, wherein the reference structure comprises a backbone of the biopolymer.

25. The method of claim 24, wherein the backbone omits at least one side chain of the biopolymer.

26. The method of claim 1, further comprising:
concurrently or sequentially altering the one or more associated biopolymer sequences to modulate one or more biophysical properties or pharmacodynamic properties of the one or more associated biopolymer sequences, the one or more biophysical properties or pharmacodynamic properties selected from: isoelectric point, weight, hydrophobicity, melting temperature, stability, Kₒₙ, K_{off}, or K_{d}, half-life, enzymatic function, aggregation, and functional activity.

27. The method of claim 1, wherein the one or more associated biopolymer sequences is a polypeptide.

28. The method of claim 27, wherein the polypeptide comprises one or more non-canonical amino acids.

29. The method of claim 27, wherein the polypeptide comprises one or more D-amino acids.

30. The method of claim 27, wherein the polypeptide is an antibody or antigen-binding fragment thereof, and the reference structure is an antibody-antigen complex.

31. The method of claim 27, wherein the polypeptide is a ligand or receptor, and the reference structure is a ligand-receptor complex.

32. The method of claim 27, wherein the polypeptide is an enzyme or substrate, and the reference structure is an enzyme-substrate complex.

33. The method of claim 1, wherein the method can provide one or more n-mer biopolymer sequences in under 3 seconds, wherein n is greater than 500.

34. The method of claim 1, wherein the one or more associated biopolymer sequences is a protein and wherein the model neural network was trained: using an ensemble of 1000, 2000, 3000, 5000, 10000, 50000, 100000, 500000, or 1000000 more protein structures, e.g., some (e.g., 10, 20, 30, 40, 50, 60, 70, 80, 90, 95%) or substantially all of the structures from the Protein Data Bank (PDB).

35. The method of claim 1, further comprising: training on the target complex, wherein the target complex involves multiple chains.

36. The method of claim 1, wherein the one or more associated biopolymer sequences are proteins and the energy landscape is a conditional random field.

37. The method of claim 1, wherein the edges are initialized using edge features based on geometric and structural relationships between the biopolymer.

38. A method comprising providing one or more associated biopolymer sequences to conform to a reference structure, the reference structure comprising a target complex, the associated biopolymer sequences obtainable by a method comprising:
obtaining a first biopolymer sequence from an energy landscape, the energy landscape generated based on a graph representation embedded using a neural network, the graph representation featurized from the reference structure and comprising a topology of biopolymer sequences as nodes and interactions between monomers as edges; and
generating one or more additional biopolymer sequences using the energy landscape, free of using the graph representation.

39. The method of claim 38, further comprising synthesizing the one or more additional biopolymer sequences.

40. The method of claim 38, further comprising contacting the one or more additional biopolymer sequences with an analyte, and wherein the analyte is a biological fluid.

41. The method of claim 38, further comprising producing one or more of the additional biopolymer sequences.

42. The method of claim 41, wherein the produced one or more of the additional biopolymer sequences is an antibody.

43. The method of claim 38, further comprising administering to a subject in need a particular biopolymer sequence, the particular biopolymer sequence being a given biopolymer sequence from amongst the first biopolymer sequence obtained and the one or more additional biopolymer sequences generated.

44. A non-transitory, computer-readable medium comprising instructions to be performed by a microprocessor, suitable for;
obtaining a first biopolymer sequence from an energy landscape, the energy landscape generated based on a graph representation embedded using a neural network, the graph representation featurized from a reference structure and comprising a topology of biopolymer sequences as nodes and interactions between monomers as edges; and
generating one or more additional biopolymer sequences using the energy landscape, free of using the graph representation.

45. A system comprising a processor and a non-transitory, computer-readable medium including instructions which, when loaded and executed by the processor, cause the system to:
obtain a first biopolymer sequence from an energy landscape, the energy landscape generated based on a graph representation embedded using a neural network, the graph representation featurized from a reference structure and comprising a topology of biopolymer sequences as nodes and interactions between monomers as edges; and
generate one or more additional biopolymer sequences using the energy landscape, free of using the graph representation.

46. A biopolymer sequence produced by:
embedding a graph representation using a neural network, the graph representation featurized from a reference structure and comprising a topology of a biopolymer with monomers as nodes and interactions between monomers as edges;
processing the graph representation with a graph neural network or equivariant neural network that iteratively updates node and edge embeddings with a learned parametric function;
converting the embedded graph representation to an energy landscape using a decoder; and
obtaining the biopolymer sequence from the energy landscape.

47. The biopolymer sequence of claim 46, wherein the biopolymer sequence is at least one of: a polypeptide and an antibody.
